(19) United States
(12) Patent Application Publication
(10) Pub. No.: US 2005/0042186 A1
(43) Pub. Date: Feb. 24, 2005

(54) All natural sunscreen lotion

(76) Inventor: Peter Zahner, Bonita Springs, FL (US)

Correspondence Address: Peter Zahner;Suite 203, 27725 Old 41 Road, Bonita Springs, FL, 34135, US

(21) Appl. No.: 10/643,270

(22) Filed: Aug. 19, 2003

(51) Int. Cl. A61K 7/42; A61K 7/06; A61K 35/78

(52) U.S. Cl. 424/59; 424/74; 424/729

(57) ABSTRACT

The present invention relates to an all natural topical sunscreen composition with an aqueous phase in the form of a lotion. The composition includes: 1) From 0.01% to 5% by weight of a (biological) melanin. 2) From 2% to 10% by weight of a green tea extract with a polyphenol content greater than 40%. 3) From 2% to 10% by weight of mineral pigments of either titanium dioxide and/or zinc oxide. Preferred embodiments are oil-in-water wherein the particular active ingredients have been pre-dispensed in synergistically effective amounts to provide the desired SPF, cell protection from free radicals and a level of water resistance.

### CROSS REFERENCE TO RELATED APPLICATIONS

(none)

### STATEMENT REGARDING FED SPONSORED R & D

(none)

### FIELD OF THE INVENTION

The invention relates to compositions, which provide dual Protection for the human skin against the damaging effects of UVA and UVB radiation. The protection encompasses: 1) UV reflection by mineral pigments and 2) cell protection by scavenging of UV induced unstable free radicals. In particular, the invention relates to all natural, absolutely chemical free products which act synergistically as an effective UVB/UVA reflector (Mineral Pigments), UVA absorber and superior free radical trap (Melanin) and cell protector against erithema and inflammation (Green Tea) in such compositions.

### BACKGROUND OF THE INVENTION

The skin is the most environmentally-stressed organ in human beings. Not only is the skin subjected to toxic chemicals and hostile environments, but it also is the only organ directly exposed to ultraviolet “UV” light in the presence of oxygen.

It has now been well established that long-term exposure to the sun increases the risk of a number of undesirable skin conditions. Not only has solar radiation been implicated as a causative agent of a number of skin cancers. It has also been shown to accelerate the aging process.

While it seems well established that excessive sun exposure will produce serious skin damage and increase the risks for certain skin cancers. The effects of overexposure to the, resulting specifically from UV radiation, resulting from short-term effects from UVB radiation, such as erithema (sunburn) to long-term effects from UVB causing premature skin aging and skin cancer.

On the other hand, it is known that we need some minimum amount of sunlight to maintain good health and a positive mental state. Sunlight produces vitamin D in the skin which is absolutely necessary for the efficient absorption of calcium and phosphate for strong bone reformation. Most of this is obtained primarily through synthesis by UVB radiation in the skin.

### SUMMARY OF THE INVENTION

The present invention relates to a sunscreen composition comprising as its active components, in synergistically effective amounts, 1) biological Melanin Extract 2) Green Tea Extract from the tea plant Camellia Oleifera or Camellia Sinensis with Polyphenol contents of greater than 40%, and 3) Titanium Dioxide in the amount of 2%-10% and/or Zinc Oxide. It has been found that when adding (Bio) Melanin Extract and Green Tea Extract, in relative small quantities, to a traditional sunscreen (Titanium Dioxide or Zinc Oxide), the combination modestly boosts the SPF by about 3 units but it provides unexpectedly high UVA protection. The content of about 1% of Melanin leads to an unexpected UVA protection, i.e. Phototoxic Protection Factor (PPF) of about 6 and will generate a pleasant immediate tan (Immediate pigment Darkening).

### DETAILED DESCRIPTION OF THE INVENTION

All ingredients of the present composition are all Natural and Nonchemical in that they are found in naturally occurring material and are routinely prepared from such materials or are readily purchased from commercial sources. For example, (Bio) Melanin is found in a number of different plants and fish extracts. It is found in the fruits of the date palm (phytomelanin) and the common cuttle fish (eumelanin). A description of the methods for the preparation of Melanin

Extract is available from Mel Co R.

For the purpose of this invention, it is important that the composition contains typically 1% of Melanin to achieve the desired UVA protection. It is known that Green Tea is high in polyphenolic antioxidants which are effective in reducing erythema response both to UVA and to UVB radiation.

For the purpose of this invention, it is important that the polyphenols content of the Green Tea Extracts should be 40%, more preferably at 50% and more. The effective amount of Titanium Oxide or Zinc Oxide, the particles in a non-surface treated form (no aluminum stereate, no aluminum hydroxide and no silica) in order to keep the formula All Natural and Chemical Free. The effective amount of TiO2 and ZnO should preferably not exceed 8% by weight in order to control the whitening effect. Adding Green Tea and (Bio) Melanin increases the SPF by 2 to 4 units. With the combination of this invention, an SPF of 18 to 20 can be achieved using only natural ingredients

Titanium Dioxide and Zinc Oxide are widely known as powerful (physical) UV reflectors in the UVB spectrum but are less efficient in the UVA area. Short-wave solar rays (UVB) are more potent in producing sunburn and are considered the main cause of basal and squamous cell carcinomas.

The long-wave solar rays (UVA) penetrate the skin more deeply and are considered the chief culprit behind wrinkling, leathering and other aspects of photoaging. The latest studies show that UVA not only increases UVB's cancer causing effects, but also may directly cause some skin cancers, including melanomas.

It is the main purpose of this invention to provide a solution to boost the protection against the damaging of UVA radiation in an all natural way beside maintaining the high level of protection against UVB rays.

Adding only 1% of (Bio) Melanin leads to an unsurpassed UVA protection of about 6 PPF (Photoxic Protection Factor) and to a pleasant immediate tan i.e. acceleration of the body's own melanin production.

It is further a purpose of this invention to demonstrate that it is more beneficial to the human body to topically apply such an All Natural sunscreen formula with an SPF 18 and a 95% UVB protection than go higher with adding chemical ingredients and reaching only 3% more. Recent in vitro and in vivo studies show the growing concern regarding side effects (skin cancer, strong estrogenic activity) caused by chemical ingredients in sunscreen products. The following illustrates asunscreen formulation comprised of the components of the invention.

| | | % |
|---|---|---|
| Ingredients | INCI Name | by weight |
| Water | Aqua | 38.0 |
| Sunflower Oil | Sunflower (Helianthus Annuus) | 10.0 |
| | Seed Oil | |
| Vegetable Glyceriqn | Glycerin | 8.5 |
| Titanium Dioxide | Titanium Dioxide | 7.5 |
| Green Tea Extract | Green Tea (Camellia Oleifera) Extract | 6.0 |
| Sweet Almond Oil | Sweet Almond (prunus dulcis) Oil | 4.0 |
| Coconut Oil | Coconut (Coco Nucifera) Oil | 4.0 |
| Jojoba Oil | Jojoba (Buxus Chinesis) Oil | 3.5 |
| Vitamin E Oil | Tocopherol | 3.5 |
| Cocoa Butter | Cocoa (Theobroma Cacao) Butter | 3.0 |
| Orange Blossom Oil | Orange Blossom (Citrus Aurantium) Oil | 2.5 |
| Rice Bran Oil | Rice Bran (Oryza Sativa) Oil | 2.0 |
| Lanolin Oil | Lanolin Oil | 1.5 |
| Xanthan Gum | Xanthan Gum | 1.5 |
| Sodium | Glycino Amino Acid | 1.0 |
| Hydroximethyl- | | |
| glycinate | | |
| Lecithin | Lecithin | 1.0 |
| (Bio) Melanin | Melanin | 1.0 |
| Eucalyptus | Eucalyptus Citriodora Oil | 1.0 |
| Citriodora Oil | | |
| Vitamin B2 | Riboflavin | 0.5 |

1. A sunscreen composition comprising as its active components in synergistically effective amounts:
a) (Bio) Melanin extract,
b) Green Tea with a polyphenol content greater than 40%,
c) Mineral Pigments

2. The sunscreen composition of claim 1, wherein said mineral pigments is Titanium Dioxide.

3. The sunscreen composition of claim 1, wherein said mineral pigments are zinc oxide.

4. The sunscreen composition of claim 1, in which said extract is present in the amount from about 0.01 to 5% by weight of the total composition.

5. The sunscreen composition of claim 1, where said melanin in the amount of 1% by weight provides a Phototoxic Protection Factor of about 6 units.

6. The sunscreen composition of claim 1 wherein said green tea component is present in an amount of 2% o 10% by weight of the total composition.

7. The sunscreen composition of claim 6 including a camellia oleifera extract.

8. The sunscreen composition of claim 6 including a camellia sinensis extract

9. The sunscreen composition of claim 6, wherein said green tea polyphenols have a ratio of extraction greater than 40%.

10. The sunscreen composition of claim 9, wherein said green tea polyphenols (GTP) have synergistic photoprotective effects on skin when combined with either titanium dioxide or zinc oxide.

11. The sunscreen composition of claim 10, wherein said GTP's are effective at reducing an erythema response (skin reddening), both to UVB and UVA radiation.

12. The sunscreen composition of claim 1, wherein said mineral pigments are active UVA/UVB reflecting sunscreen ingredients.

13. The sunscreen composition of claim 12, wherein titanium dioxide is one of the mineral pigments is present in said composition in an amount of 2% to 10%.

14. The sunscreen composition of claim 12, wherein zinc oxide as one of the mineral pigments is present in said composition in an amount of 2% to 10%.

15. The sunscreen composition of claim 12, wherein said mineral pigments provide a UVB protection of 75% for SPF 4, 88% for SPF 8 and 93% for SPF 15.

16. A method of protecting the skin against damage from UVA and UVB radiation comprising the steps of applying a protective composition on said skin:
said composition having been prepared by active components in synergistically effective amounts:
a) using a (biological) melanin extract;
b) adding a green tea extract from the tea plant camellia oleifera or camellia sinensis with a polyphenol contents of greater than 40%
c) adding mineral pigments in the amount of 2% to 10%.

17. The method of claim 16, wherein said above components a) through c) provide dual protection against skin damages from UV radiation:
1) UV reflection and
2) cell protection by scavenging of UV induced unstable tree radicals.

18. The method of claim 16, wherein said mineral pigments reflect UVA and UVB rays on the skin.

19. The method of claim 16, wherein said bio melanin is a UV absorber and provides a significant UVA protection.

20. The method of claim 16, wherein said bio melanin acts as a dynamic antioxidant and a free radical scavenger.

21. The method of claim 16, wherein said bio melanin extract is a superior free radical trap and capable to regenerate the neutralized traditional antioxidant (polyphenols) green tea.

22. The method of claim 16, wherein said polyphenols of said green tea are efficient at protecting against erythema.

23. The method of claim 16, in which the particles of said mineral pigments remain untreated in order to keep said composition in all natural state.
